(19) United States
(12) Patent Application Publication
(10) Pub. No.: US 2015/0051095 A1
(43) Pub. Date: Feb. 19, 2015

(54) METHOD OF USING DNA STRUCTURE

(71) Applicant: Samsung Electronics Co., Ltd., Suwon-si (KR)

(72) Inventors: Chang-won LEE, Hwaseong-si (KR); Sung-ha PARK, Gwacheon-si (KR); Jun-young SON, Suwon-si (KR); Jun-wye LEE, Seoul (KR)

(73) Assignee: Samsung Electronics Co., Ltd., Suwon-si (KR)

(21) Appl. No.: 14/192,961

(22) Filed: Feb. 28, 2014

(30) Foreign Application Priority Data
Aug. 14, 2013 (KR) 10-2013-0096882

(51) Int. Cl. C12Q 1/68

(52) U.S. Cl. CPC C12Q 1/6837
USPC 506/9; 435/6.11

(57) ABSTRACT

A method of using a DNA structure includes forming a DNA structure including a plurality of spaces; attaching a DNA building block to the spaces; contacting the DNA building block with a clinical specimen; and separating the DNA building block from the DNA structure. After the DNA building block is separated from the DNA structure, the method may further include attaching a new DNA building block to the spaces. The DNA building block includes a first DNA strand attached to the spaces; and a second DNA strand attached to the first DNA strand.

### CROSS-REFERENCE TO RELATED APPLICATIONS

This application claims the benefit of Korean Patent Application No. 10-2013-0096882, filed on Aug. 14, 2013, in the Korean Intellectual Property Office, the disclosure of which is incorporated herein in its entirety by reference.

### BACKGROUND

1. Field

The present disclosure relates DNA structures, and in particular, to methods of re-using DNA structures.

2. Description of the Related Art

DNA has a double helix structure, each helix consisting of chemical structure units referred to as nucleotides which is comprised of a base, a ugar (deoxyribose), and at least one phosphate group. There are four nucleobases: adenine (A), cytosine (C), guanine (G), and thymine (T). The sequence of nucleobases, that is, the base sequence, determines genetic information which directs the functions of a living thing. Protein and generic information may vary with the base sequence.

Recently, a human genome project for encoding the base sequence of the entire human genome has been completed, which has led to a substantial development in the diagnosis and treatment of intractable diseases by using genes. Accordingly, an era of, so called, customized or personalized medicine has opened.

A molecular biological gene examination using DNA may be employed to identify the existence and amount of a particular gene in a specimen such as a clinical sample. Thus, a disease may be rapidly and accurately diagnosed.

Examples of the molecular biological gene examination methods are a polymerase chain reaction (PCR), blotting, and hybridization. Recently, DNA chips are getting attention.

Such gene examination methods are carried out using various detection kits, which are, in general, disposable.

### SUMMARY

Provided are methods of using a DNA structure, the methods decreasing resource consumption and diagnosis costs.

Additional aspects will be set forth in part in the description which follows and, in part, will be apparent from the description, or may be learned by practice of the presented embodiments.

According to an examplary embodiment, a method of using a DNA structure includes: forming a DNA structure including a plurality of spaces; attaching a DNA building block to the spaces; contacting the DNA building block with a clinical specimen; and separating the DNA building block from the DNA structure.

The method may further include, after the DNA building block is separated, attaching a new DNA building block to the spaces.

The DNA building block may include: a first DNA strand attached to the spaces; and a second DNA strand attached to the first DNA strand.

The separating of the DNA building block from the DNA structure may include annealing the DNA structure with the DNA building block attached thereto.

The forming of the DNA structure may include: placing a substrate in a 1×TAE/Mg²⁺ buffer [Tris-Acetate-EDTA (40 mM Tris, 1 mM EDTA (pH 8.0), 12.5 mM Mg(Ac)₂)].

The second DNA strand may include a material that has a base complementary to a material included in a clinical specimen. The material that has a base complementary to a material included in a clinical specimen may be biotin or an SH functional group.

### BRIEF DESCRIPTION OF THE DRAWINGS

These and/or other aspects will become apparent and more readily appreciated from the following description of the embodiments, taken in conjunction with the accompanying drawings in which:

FIGS. 1 to 4 shows front views illustrating a method of using a DNA structure according to an embodiment; and

FIG. 5 shows an example of the DNA structure illustrated in FIGS. 1 to 4 used together with a different stack structure on a substrate.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

Hereinafter, a method of using a DNA structure according to embodiments will be described in detail by referring to the attached drawings. In the drawings, thicknesses of illustrated layers or regions are exaggerated for clarity.

A two-dimensional periodic lattice formed of coplanar repeating units, each of which is composed of at least two antiparallel nucleic acid multi-crossover molecules and a three-dimensional periodic lattice which may be formed as an extension of the two-dimensional period lattice into a third dimension, such as by interconnecting adjacent two-dimensional lattices by joining together antiparallel nucleic acid multi-crossover molecules in adjacent planes are disclosed in, for example U.S. Pat. No. 6,255,469 to Seeman. The content of U.S. Pat. No. 6,255,469 is incorporated herein by reference.

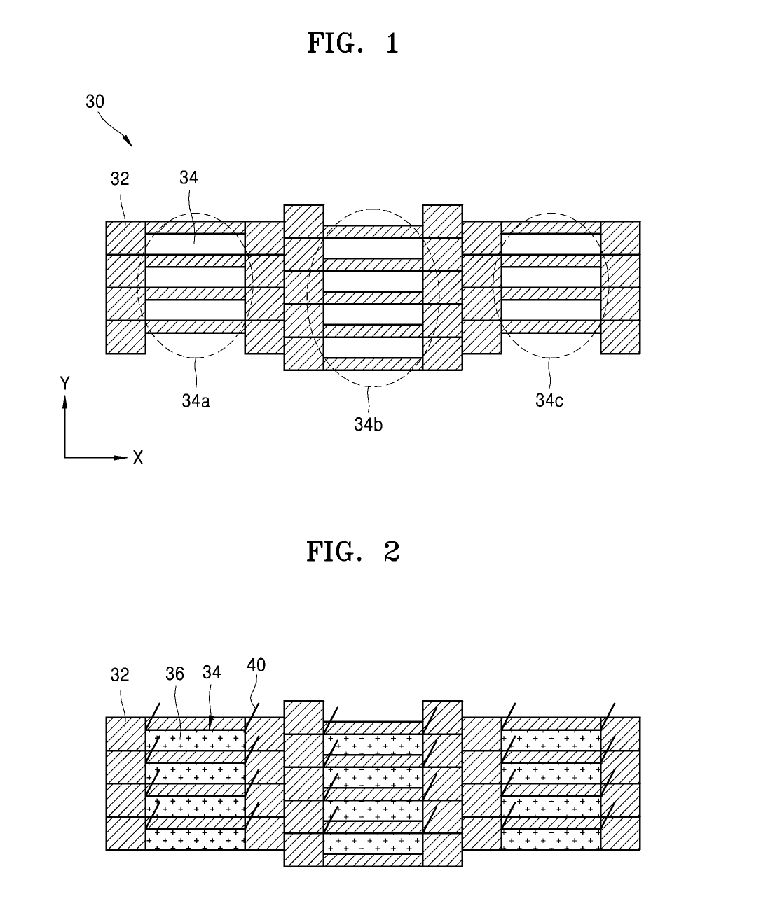

FIG. 1 illustrates a DNA structure 30 used for a method of using a DNA structure according to an exemplary embodiment.

Referring to FIG. 1, the DNA structure 30 includes a DNA frame 32 consisting of at least two DNA multi-crossover molecules; and a plurality of spaces 34, wherein the spaces 34 exist in the DNA frame 32. The spaces 34 may be periodically arranged in a Y-axis direction at constant intervals. Space groups 34a, 34b, and 34c, each consisting of spaces 34 arranged in the Y-axis direction, are arranged in an X-axis direction at certain intervals. The number of the spaces 34 along the X-axis and Y-axis directions may be greater than the number illustrated in FIG. 1. That is, the DNA structure 30 is not limited to the structure illustrated in FIG. 1, and the total size or the number of the spaces 34 may increase or decrease according to purpose.

The DNA structure 30 is formed on a substrate (not shown in FIG. 1) by placing the substrate in a 1×TAE/Mg²⁺ buffer [Tris-Acetate-EDTA (40 mM Tris, 1 mM EDTA (pH 8.0), 12.5 mM Mg(Ac)₂)] aqueous solution for a certain period of time by employing a known method disclosed in, for example U.S. Pat. No. 6,255,469. The substrate may be, for example, a silicon substrate.

Then, as illustrated in FIG. 2, a first DNA strand 36 may be attached to each of the spaces 34 of the DNA frame 32. When the DNA structure 30 is formed, ends of each of the spaces 34 may each include a sticky-end including five or more bases. The bases of the sticky-end may be complementary to those of the first DNA strand 36 in consideration of an attachment of the first DNA strand 36. Accordingly, only the first DNA strand 36 that has bases complementary to the bases of the sticky-end may be attached to each of the spaces 34 of the DNA frame 32 The first DNA strand 36 is attached to each of the spaces 34 of the DNA structure 30 as follows. The DNA structure 30 is added to an aqueous solution including the first DNA strand 36, and then, the resultant solution is annealed at, for example, about 40° C.

Following the attachment of the first DNA strand 36, a second DNA strand 40 is attached to each side of the first DNA strand 36. The first and second DNA strands 36 and 40 may be designed such that bases of the first DNA strands 36 and bases of the second DNA strands 40 are complementary to each other to attach the first and second DNA strands 36 and 40. The second DNA strand 40 may be attached to the first DNA strand 36 as follows. The DNA structure 30 with the first DNA strand 36 attached thereto is added to an aqueous solution including the second DNA strand 40, and then, heated to a certain temperature.

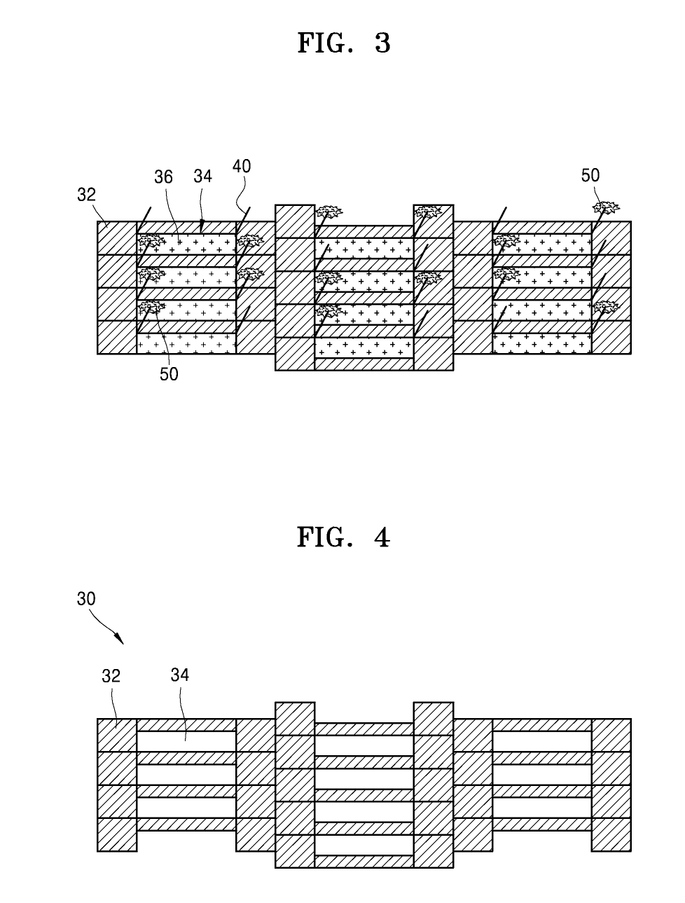

The second DNA strand 40 may be designed to have a sticky-end including a base complementary to a target nucleic acid contained in a sample to be tested. Also, the second DNA strand 40 may be coupled to a biotin-streptavidine label. The biotin-streptavidin may be coupled to gold nanoparticles. The second DNA strand 40 may include a SH functional group to which gold nanoparticles or biotin-streptavidine-gold nanoparticles are attached. As such, only a target nucleic acid that has a base complementary to the second DNA strand 40 may be attached to the second DNA strand 40. Accordingly, by bringing the DNA structure 30 with the second DNA strand 40 attached thereto in contact with a sample such as a biological sample or a clinical specimen, it may be possible to identify that the sample includes a target sequence that is a base complementary to the second DNA strand 40. When the clinical specimen includes a material that has a base complementary to the second DNA strand 40, as illustrated in FIG. 3, a material 50 that has a base complementary to the second DNA strand 40 is attached to the second DNA strand 40.

Thereafter, when the material 50 is attached to the second DNA strand 40, the DNA structure 30 is annealed at a predetermined temperature. The annealing temperature may be any temperature at which the complementary bond of base pairs of the sticky-end of each of the spaces 34 and the first DNA strand 36 is broken. An annealing temperature and time appropriate for cutting a base pair are well known. The annealing temperature may be adjustable according to external environmental factors of the DNA structure 30.

Due to the annealing, the first DNA strand 36 is separated from the spaces 34, and ultimately, the first and second DNA strands 36 and 40 are separated from the DNA frame 32. Thus, as illustrated in FIG. 4, the remaining DNA structure 30 has the spaces 34 from which the first and second DNA strands 36 and 40 are separated. The DNA structure 30 of FIG. 4 may also be re-used as described in connection with FIGS. 2 and 3. The first and second DNA strands 36 and 40 are also referred to as DNA building blocks. First and second DNA strands 36 and 40 attached to respective repeating unit (or DNA frame) of the DNA structure may be the same or different.

According to an examplary embodiment, when the DNA structure 30 is used as explained in connection with FIGS. 1 to 4, although not illustrated, the DNA structure 30 may be attached to a substrate. The substrate may be a silicon substrate, and may include a transistor to be connected to the DNA structure 30.

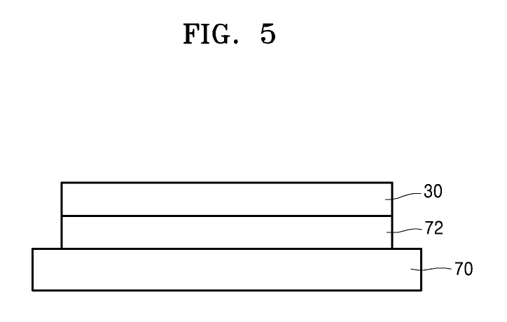

The DNA structure 30 may not be directly disposed on a substrate 70, and as illustrated in FIG. 5, may be disposed on a stack structure 72 disposed on the substrate 70. The stack structure 72 may be a DNA layer with no spaces therein.

As described above, a method of using a DNA structure according to the one or more of the above example embodiments may be appropriate for a multiple re-use of the DNA structure. Accordingly, consumption of resources and costs for forming the DNA structure may decrease.

Also, in the case of a disposable kit used in the related art, as the number of diagnosis increases, more examination kits are needed. However, when a method of using a DNA structure according to example embodiments is used, a multiple use of a single DNA structure is possible. Accordingly, it is possible to reduce articles to prepare for diagnosis.

It should be understood that the exemplary embodiments described therein should be considered in a descriptive sense only and not for purposes of limitation. Descriptions of features or aspects within each embodiment should typically be considered as available for other similar features or aspects in other embodiments.

What is claimed is:

1. A method of using a DNA structure, the method comprising:
forming a DNA structure including a plurality of spaces;
attaching a DNA building block to the spaces;
contacting the DNA building block with a clinical specimen; and
separating the DNA building block from the DNA structure.

2. The method of claim 1, further comprising:
After the DNA building block is separated, attaching a new DNA building block to the spaces.

3. The method of claim 1, wherein
the DNA building block includes:
a first DNA strand attached to the spaces; and
a second DNA strand attached to the first DNA strand.

4. The method of claim 1, wherein
the separating of the DNA building block from the DNA structure includes annealing the DNA structure with the DNA building block attached thereto.

5. The method of claim 1, wherein
the forming of the DNA structure includes:
placing a substrate in a 1×TAE/Mg²⁺ buffer comprising 40 mM Tris, 1 mM EDTA of pH 8.0, and 12.5 mM Mg(Ac)₂).

6. The method of claim 3, wherein
the second DNA strand includes a material that has a base complementary to a material included in a clinical specimen.

7. The method of claim 6, wherein
the second DNA strand has a SH functional group.
